(19) United States
(12) Patent Application Publication
(10) Pub. No.: US 2009/0156891 A1
(43) Pub. Date: Jun. 18, 2009

(54) Prolapse and Perineal Repair Concepts

(75) Inventors: Amanda J. Heys, Eden Prairie, MN (US); Karen Pilney Montpetit, Mendota Heights, MN (US); David J. Kupiecki, Edina, MN (US); John A. Zawacki, Shorewood, MN (US); Daniel Taut, Eden Prairie, MN (US); Shawn Michael Wignall, Woodbury, MN (US); Gregory L. Koeller, Apple Valley, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/334,192

(22) Filed: Dec. 12, 2008

Related U.S. Application Data
(60) Provisional application No. 61/013,237, filed on Dec. 12, 2007.

(51) Int. Cl. A61F 2/02

(52) U.S. Cl. 600/37

(57) ABSTRACT

A pelvic implant assembly having a support member and an insertion tool. The insertion tool having an implant support portion for supporting the support member as it is being inserted into a tight therapeutic location within a patient. The support member is coupled to and extends between the implant support portions prior to insertion into the incision. Once the insertion tool and accompanied support member are in the therapeutic location the insertion tool can open and spread out the support member. The insertion tool can also dilate the tissue as it opens, thereby, eliminating the need to dissect tissue.

### PRIORITY CLAIM

The present application claims the benefit of U.S. Provisional Application No. 61/013,237 filed Dec. 12, 2007, which is incorporated herein in its entirety by reference.

### FIELD OF THE INVENTION

The present invention relates to devices and methods of treating pelvic conditions and particularly to devices and methods for treating incontinence and prolapse conditions.

### BACKGROUND

Fecal incontinence is a common problem that occurs in both men and women. In women it is often occurs after vaginal childbirth, presumably the result of trauma to pelvic floor muscles, supporting fascia and nerves. Fecal incontinence affects an estimated 7.6 percent of women between the ages of 30-90. The prevalence increases with age, affecting 3.6 percent of women between 30-39 and 15.2 percent of women between 80-90. Several anatomical features contribute to fecal continence, including the resting tone of the external and internal anal sphincters, as well as the position of the levator ani muscles, especially the puborectalis muscle, which forms a sling around the rectum and is responsible for the so-called “ano-rectal angle,” which keeps stool in the rectum until voluntary defecation relaxes the puborectalis muscle and straightens the angle, allowing stool to move towards the anus.

Fecal incontinence may occur as the result of several mechanisms, including direct damage to the internal or external anal sphincters (from iatrogenic episiotomy or spontaneous lacerations during vaginal delivery), or to the levator ani muscles. It may also result from indirect injury of these muscles through denervation of the nerves that supply these muscles. Treatment of this problem has centered on pelvic floor rehabilitation, dietary changes, or surgical correction. Surgery has been used to treat specific defects in the anal sphincters, such as external anal sphincteroplasty. Success rates of only 50% or less are generally reported for these procedures on long-term follow-up.

Recently, an artificial anal sphincter has been used to bypass these muscles, though this surgery involves fairly extensive dissection and requires the patient to depress a subcutaneous valve which temporarily deflates the sphincter cuff and allows voluntary defecation. This procedure is performed in very few centers in the U.S., and even in experienced hands, complications occur frequently. Dynamic graciloplasty, which involves mobilization and wrapping of the gracilis muscle around the anorectum is now another accepted procedure although it remains complex and requires extensive experience to obtain good results.

In addition, many women report other symptoms of bowel dysfunction, such as constipation and incomplete bowel emptying. For some women, these symptoms are due to either an anterior rectocele (a hernia of the rectum into the vaginal canal), or due to a defect in the levator ani muscles, which results in descent of the levator plate and/or perineum with abdominal straining. In addition, patients may be noted to have a defect in the posterior aspect of the rectum, or a posterior rectocele. There are very few treatment options for this condition, though retrorectal levatorplasty has been used in the past. In this procedure, an incision is made between the anus and the coccyx and the levator muscles are exposed bilaterally. Sutures are then placed in the levator muscles to plicate them together in the midline.

Pelvic organ prolapse is a condition where organs, such as the uterus, the rectum, or the bladder, fall down or slip out of place within a person's body. It is commonly used in reference to organs protruding through a woman's vagina, but prolapse may occur within men as well. In general, the levator ani muscles provide the main support for pelvic organs including, for example, the rectum, the vagina, and the urethra. In a person with a normal pelvis, the levator ani muscles keep the pelvic floor closed, thereby allowing the pelvic and abdominal organs to rest on the levator ani muscles. This also significantly reduces the tension that would otherwise be placed on the fascia and ligaments that support the pelvic organs. The posterior portion of the levator ani muscles arise from the area of the tendinous arch. The anterior portion of the levator ani muscles arise from the superior pubic rami and from the anterior end of the obturator internus muscles. This portion forms the pubococcygeus muscle and the puborectalis muscle. The pubococcygeus muscle is a generally thick, U-shaped muscle through which the urethra, vagina, and rectum transverse. The pubococcygeus muscle supports these structures at rest, and helps to augment the endopelvic fascia during coughing or straining. The puborectalis muscle is structured to act as a sling support for the rectum, and includes two ends that attach to the anterior side of the symphysis pubis. The levator ani muscle group includes an opening through which the vagina and urethra pass, which is referred to as the urogenital hiatus. Similarly, the levator ani muscle group includes an opening through which the rectum passes, which is referred to as the rectal hiatus.

In a normal woman, the pelvic floor muscles support most of the weight of the pelvic organs, such as the vagina, uterus, bladder, and rectum. Additionally, the various pelvic fascia and ligaments stabilize these structures in position. When the normal anatomic relationships in the pelvis are disrupted, or if injuries occur, dysfunctions such as urinary incontinence, fecal incontinence, or prolapse of the pelvic organs, may occur. For example, if one of the levator ani muscles is damaged, the muscle may be unable to adequately support the weight of the pelvic organs. This will result in a disproportionate amount of the pelvic organ weight being placed onto the pelvic ligaments, which are significantly weaker than the fibrous tissue of the ligaments that connect bones. In particular, the pelvic ligaments are more accurately described as thickening of the endopelvic fascia tissue, which is composed of collagen, smooth muscle, elastin, and fibrovascular bundles. These ligaments are not designed to carry the increased load resulting from problems in the pelvic floor. As a result, these ligaments may eventually fail. The failure or damage to the pelvic floor ligaments may cause, for example, the bladder, rectum, or uterus to prolapse through the vagina. Similarly, expansion of or damage to the levator or rectal hiatus may result in the bladder, vagina, or rectum prolapsing through the hiatus.

As stated previously, pelvic prolapse conditions result from the weakness or damage to the normal pelvic-support systems. In general, the main categories of pelvic prolapse include cystocele, rectocele, enterocele, uterine prolapse, and vaginal vault prolapse. The most common causes of these pelvic floor disorders in a female patient include child birth and removal of the uterus (hysterectomy). However, other contributing factors may include connective tissue defects, prolonged heavy physical labor, postmenopausal atrophy, neurogenic weakness of muscles, muscle weakness due to aging, and obesity.

A cystocele occurs when damage to the pubocervical fascia in the central or lateral areas (or both) allows the bladder to protrude into the vagina. Simply defined, a cystocele is a protrusion of the bladder into the vagina due to defects in pelvic support.

A rectocele is a bulge into the vagina caused by the rectum prolapsing through an attenuated rectovaginal septum. A rectocele is commonly a result of childbirth or chronic constipation. During childbirth, the rectovaginal septum and surrounding vaginal tissues are stretched and disrupted, which may cause weakness and stretching in these tissues. A rectocele typically forms a pocket just above the anal sphincter where stool may become trapped.

An enterocele is essentially a herniation of the small bowel into the vagina. Specifically, the peritoneal sac containing a portion of the small bowel extends into the rectovaginal space between the posterior surface of the vagina and the anterior surface of the rectum.

A uterine prolapse is the distention of the uterus and cervix outside the vagina. It is often associated with a rectocele, cystocele, or entrocele.

Finally, a vaginal vault prolapse is the distention of the vaginal apex after hysterectomy outside the vagina. It is also often associated with a rectocele, cystocele, or entrocele.

There is a need for a device and procedure of treatment that corrects prolapse and fecal incontinence without the need for extensive surgery. There is additionally a need for a device that can place a sling in a minimal incision without dissection of a patient's tissue.

The background section of this application is provided to identify a need in the field and nothing contained within the background should be considered prior art to the examples of the applicant's invention disclosed herein.

### BRIEF SUMMARY OF THE INVENTION

The invention relates to a pelvic implant assembly comprising a support member and an insertion tool. The insertion tool comprises an implant support portion for supporting the support member as it is being inserted into a tight therapeutic location within a patient. The support member is coupled to and extends between the implant support portions prior to insertion into the incision. Once the insertion tool and accompanied support member are in the therapeutic location the insertion tool can open and spread out the support member. The insertion tool can also dilate the tissue as it opens, thereby, eliminating the need to dissect tissue.

### BRIEF DESCRIPTION OF THE DRAWINGS

FIG. 1 illustrates anatomy relevant to the invention, including implantation of a support member at specific anatomical structures.

FIG. 2 illustrates anatomy relevant to the invention, including implantation of a support member at specific anatomical structures.

FIG. 3A illustrates a support member positioned in a cavity.

FIG. 3B illustrates an insertion tool in a closed position.

FIG. 3C illustrates the insertion tool of 3B in an open position.

FIG. 3D illustrates an end view of the insertion tool of FIG. 3B.

FIG. 3E illustrates a support member.

FIG. 4 illustrates a partial view of the insertion tool and support member.

FIG. 5A illustrates an insertion tool in a closed position.

FIGS. 5B-5D illustrates an insertion tool having various shapes in an open position.

FIG. 6A illustrates an insertion tool positioned in a support member.

FIG. 6B illustrates a support member folded over an insertion tool.

FIG. 6C illustrates a support member and insertion tool positioned in a cavity.

FIG. 6D illustrates a support member in an unfolded state within a cavity.

FIG. 7A illustrates a support member having resilient members.

FIG. 7B illustrates a support member moving from a coiled position toward a generally uncoiled position.

FIG. 8 illustrates a support member disposed in a sheath.

FIG. 9 illustrates a support member folded and tied.

FIG. 10A illustrates a dilator in a closed position.

FIG. 10B illustrates the dilator of 10A in a partially open position.

FIG. 10C illustrates the dilator of 10A in an open position.

### DETAILED DESCRIPTION OF EXAMPLE EMBODIMENTS

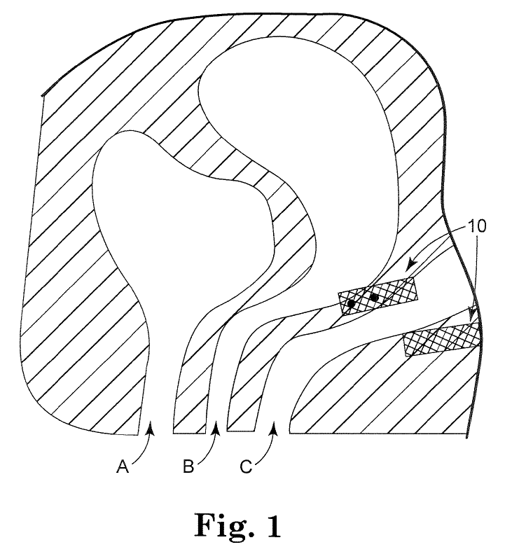

The treatment of prolapse and fecal incontinence can be treated by placing a support member 10 in a therapeutic location within a patient's pelvic cavity without the need to create large incisions or dissect large amounts of tissue. Some example locations for implantation include those identified in the drawings, including FIGS. 1 and 2, which illustrate a urethra A and a vagina B with a vaginal wall positioned therebetween. FIG. 1 also illustrates a rectum C with the support member 10 being positioned in the tissue between the vagina B and the rectum C. In another example embodiment, another support member 10 can either additionally or alternatively be placed in tissue posterior of the rectum C. Other locations are also possible and should be considered to be within the spirit and scope of the invention.

Turning to FIG. 2, a support member 10 is illustrated as being implanted in tissue that extends at least partially around a portion of the rectum C. In another example embodiment of the invention, the support member 10 can be positioned completely around a segment of a patient's rectum C. It is also possible to place a support member 10 completely or partially around other organs or structures such as a urethra, bladder, bladder neck, vagina, cervix, uterus and the like.

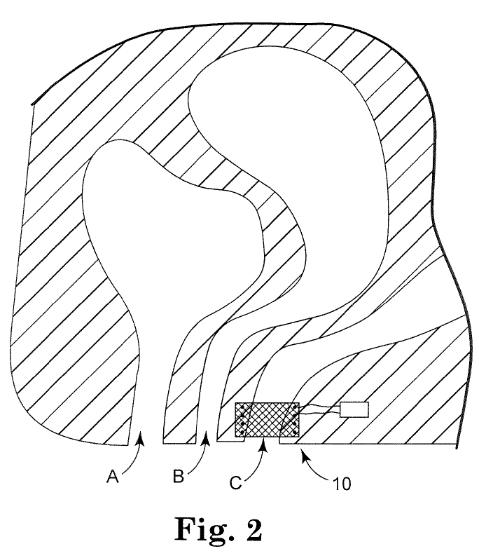

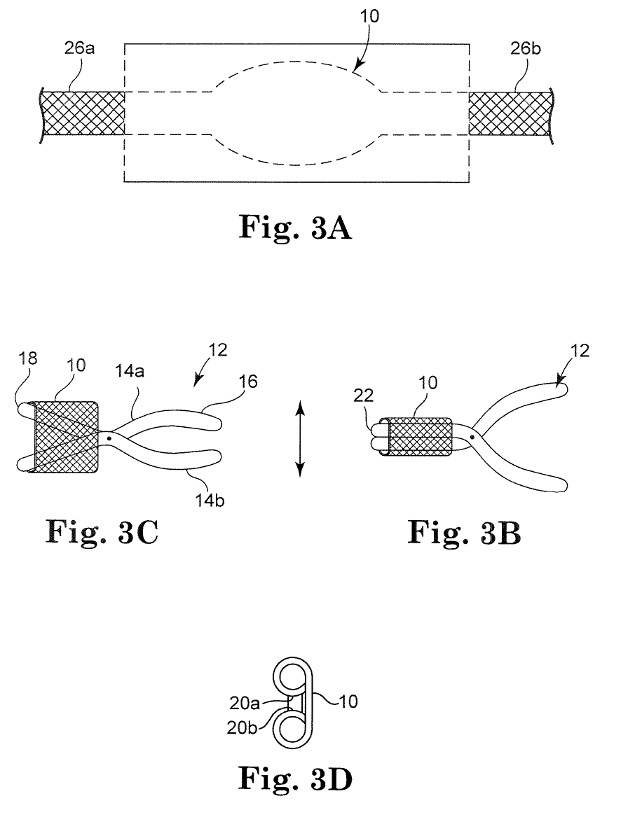

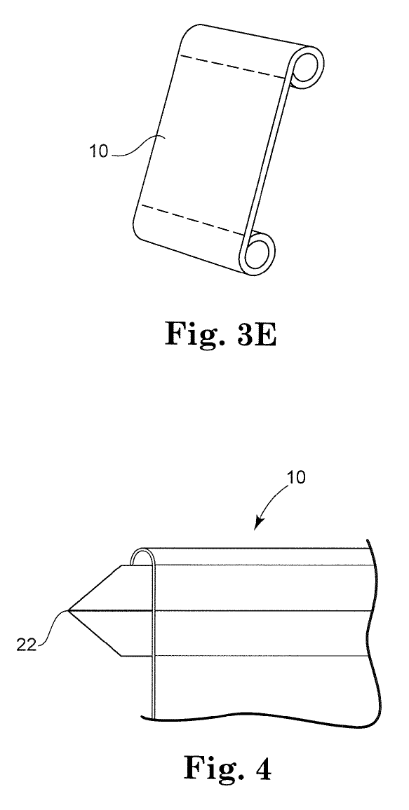

In one embodiment of the invention, the support member 10 can be operatively used with an insertion tool 12 to implant the support member 10 through one or more incisions without the need to dissect a large amount of tissue. Turning now to the series of drawings illustrated in FIGS. 3A-3E, there is shown a support member 10 disposed on an insertion tool 12 that is capable of being inserted into an incision and expanding within the patient's pelvic cavity.

The insertion tool 12, illustrated in FIGS. 3B-4, can have a pair of operatively connected arms 14a and 14b. Each of the arms 14a and 14b comprises a handle portion 16 and an implant support portion 18. The arms 14a and 14b are pivotally coupled together by a coupler that is positioned approximately between the handle portions 16 and the implant support portions 18 such that movement of the handle portions 16 translate to movement of the implant support portions 18.

As illustrated in FIGS. 3B and 3C, the insertion tool 12 is positionable between an open position and a closed position. As particularly illustrated in FIG. 3B, when the tissue support portions 18 of each of the arms 14a and 14b are proximate to each other the insertion tool 12 is considered to be in a closed position. As particularly illustrated in FIG. 3C, when the tissue support portions 18 of each of the arms 14a and 14 are positioned or spread apart from each other the insertion tool 12 is considered to be in an open position.

The tissue support portions 18 can each include retaining members (not shown) that can extend axially along a length of the tissue support portions 18. The retaining members can consist of clips or recesses or channels extending into each of the tissue support portions 18. The support member 10 can have opposed ends 20a and 20b that are wound, wrapped or coiled about a respective tissue support portion 18 and at least temporarily retained by the retaining members. As illustrated in FIGS. 3B-3C, the support member 10 can extend between the tissue support portions 18 of each of the arms 14a and 14b when ends 20a and 20b are coupled to the tissue support portions 18.

In one example embodiment, the tissue support portions 18 can include channels or recesses extending therein that permit the support member 10 to be generally flush with an outer surface of the tissue support members 18. In yet another embodiment, the tissue support portions 18 can have rollers mounted axially thereon. Each end 20a and 20b of the support member 10 can be wound about a respective roller such that they can be unwound during implantation.

Free ends 22 of each of the tissue support portions 18 can be shaped to facilitate insertion into an incision. In one example embodiment, the free ends 22 of the tissue support portions 18 can be generally blunt. Free ends 22 of each of the tissue support portions 18 can also be generally pointed. As particularly illustrated in FIG. 4, each of the free ends 22 of the tissue support portions 18 can generally taper such that when in the closed position the tissue support portions 18 generally form a point.

Returning to FIG. 3A, the support member 10 can include a central portion 24 with one or more appendages 26a and 26b extending away from the central portion 24. In this particular embodiment, the central portion 24 can be disposed on the tissue support portions 18 of the insertion tool 12 for insertion into one incision. However, multiple incisions can be made to accommodate the appendages 26a and 26b. The appendages 26a and 26b can also include connectors for connecting to a delivery device that is used to pass the appendage through the multiple incisions. A delivery device such as a helical type needle, as disclosed in U.S. Pat. No. 6,911,003 and incorporated herein in its entirety, can be used to insert the support member 10 around the external sphincter of the rectum C. Other types of needles known in the art can also be used. For example, straight and curved needles can also be used.

In any of the embodiments one or more anchors can be disposed to at least one end of the support member 10 or appendages 26a and 26 for anchoring into tissue such as muscle, ligament, adipose and the like. It is also possible to have one or more of the appendages 26a or 26b with anchors to be inserted into the patient's body and wrapped around the rectum C to provide additional support. It is also possible to have the appendages 26a and 26b wrap around the rectum C in opposite directions and anchored into the patient's tissue. In yet another embodiment, the appendages 26a and/or 26b can be cut proximal a skin incision and allowed to become fixated in the muscle or tissue.

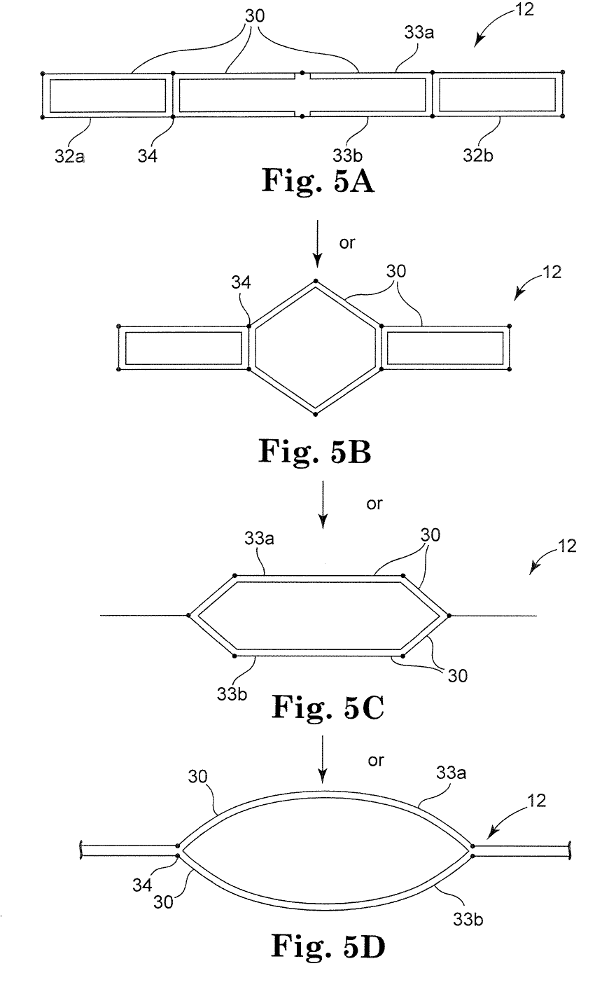

As illustrated in the series of FIGS. 5A-5D, the insertion tool 12 can comprise multiple interconnected segments or linkages 30. The insertion tool 12 of this example embodiment comprises two or more end or actuator portions 32a and 32b. Each of the actuator portions 32a and 32b is operatively coupled to two or more expansion portions 33a and 33b. The expansion portions 33a and 33b can be coupled to the actuator portions 32a and 32b by joints 34 that permit the expansion portions 33a and 33b to move relative to the actuator portions 32a and 32b. The joints 34 can consist of a ball and socket, pin and socket, living hinge and the like. Other joints or combination of joints are possible and the foregoing should not be considered limiting.

As illustrated in FIG. 5A, the insertion tool 12 has a closed position where the expansion portions 33a and 33b are relatively close to each other and the insertion tool 12 has a generally narrow or uniform width along its axis. The insertion tool 12 also has an open position, as illustrated in FIG. 5B, in which the expansion portions 33a and 33b are relatively positioned apart from each other such that the insertion tool 12 has a portion or segment that is generally wider then a width of actuator portions 32a and 32b. The insertion tool 12 can be moved from the closed position toward the open position by moving the actuator portions 32a and 32b axially toward each other, thereby, causing the expansion portions 33a and 33b to move away from each other as illustrated in FIG. 5B. To move the insertion tool 12 toward the closed position from the open position a user can pull the actuators 32a and 32b axially away from each other, thereby, causing the expansion portions 33a and 33b to come closer together as illustrated in FIG. 5A

FIGS. 5B, 5C and 5D illustrate example embodiments of the insertion tool 12 in an open position. As illustrated in FIG. 5B the expansion portions 33a and 33b can have one intermediate joint 34 such that the expansion portions 33a and 33b of the insertion tool 12 generally form a regular hexagon shape. As illustrated in FIG. 5C the expansion portions 33a and 33b can have two joints such that the expansion portions 33a and 33b of the insertion tool 12 generally form an irregular hexagon shape. Lastly, as illustrated in FIG. 5D the expansion portions 33a and 33b can have no intermediate joints 34 but rather have two expansion portions 33a and 33b that are pliable or bendable. Numerous joints and shapes are possible to control movement of the expansion portions 33a and 33b. The examples described herein should not be considered limiting.

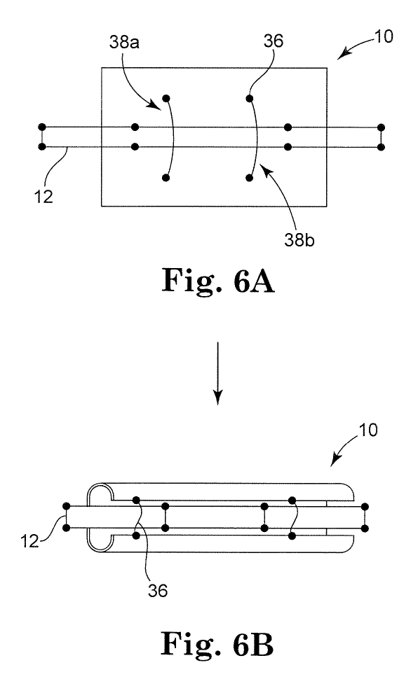

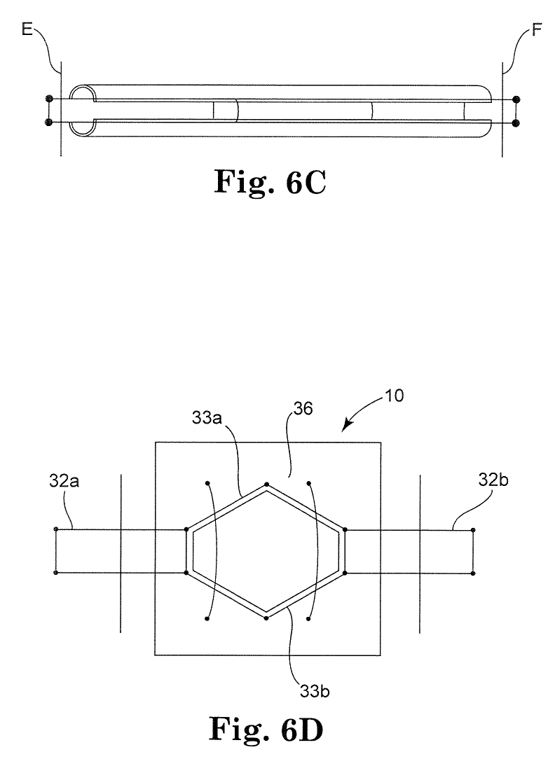

Turning now to FIGS. 6A-6D, the support member 10 can include a pocket 36 created or formed on one of its side surfaces that is capable of receiving at least a portion of the insertion tool 12. In one example embodiment the pocket 36 can be made by tying two spaced apart lengths of sutures generally parallel to each other. In another example embodiment the pocket 36 can be made my tying, heat setting or otherwise coupling another piece of material to the support member 10. As illustrated in FIG. 6A, the insertion tool 12 can be inserted through openings 38a and 38b of pocket 36. When the insertion tool 12 is properly positioned in the pocket 36 the ends of the insertion tool 12 extend out of openings 38a and 38b respectively while the expansion portions 33a and 33b are positioned within the pocket 36.

Turning now to FIG. 6B, the support member 10 can be pliable and capable of being wound, coiled, or curled about the insertion tool 12 prior to insertion into an incision. As illustrated in FIG. 6C the ends of insertion tool 12 preferably extend out of incisions E and F so that a physician can move the actuators 32a and 32b to move the expansion portions 33a and 33b from the closed position toward the open position. As the expansion portions 33a and 33b move toward the open position they can dilate the tissue, if not done earlier, uncoil the support member 10 and permit it to lay flat against the desired organ or tissue. Once a physician has determined that the support member 10 is properly positioned the actuators 32a and 32b can be moved away each other, thereby, causing the expansion portions 33a and 33b to move toward the closed position. The insertion tool 12 can then be removed and the incisions closed.

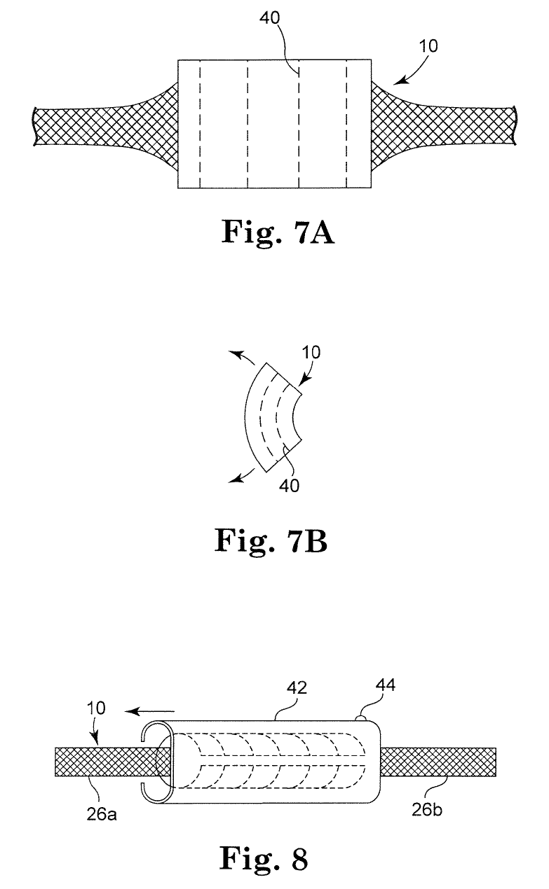

In another example embodiment of the invention, as illustrated in FIGS. 7A and 78, the support member 10 can include resilient members 40 attached to or formed part of the support member 10. The resilient members 40 can be disposed along a portion or the entire length or width of the support member 10. The resilient members 40 can be disposed in any configuration to move the support member 10 from a coiled, folded, bent or similar state to an open generally flat state. It is also possible to have resilient members 40 that have a natural at least semi-coiled state. This would permit the support member 10 to generally coil about an anatomical structure. The resilient members 40 can comprise a pliable plastic, nitinol, a polymer or a low plastically deforming metal. Other materials, known or yet developed, having similar properties should be considered to be within the spirit and scope of the invention.

To implant this particular example embodiment, the insertion tool 12 can comprise a sheath or tube 42 capable of transporting the support member 10 into position within a patient's pelvic cavity. The sheath 42 can be a generally rigid or pliable material such as a polymer, nitinol, or a plastically deforming metal. Examples of sheaths 42 currently being used by Applicant and which can be used with this invention include those used to cover the arms of the Monarc® mesh sling. Other materials are also contemplated and the above should not be considered limiting.

As illustrated in FIG. 8, the support member 10 can be coiled or folded and placed into the sheath 42. The sheath 42 can then be inserted into an incision. In one example embodiment, one or more couplers 44 can be disposed on or operatively coupled to the sheath 42 for coupling to a needle or similar deliver device to facilitate passage through the incision and into a therapeutic location for implantation. Once in place the sheath 42 can be removed exposing the support member 10. The resilient members 40 then cause the support member 10 to uncoil or unfold toward its natural state, which could be flat or slightly coiled. If the support member 10 has arm portions 26a and 26b they can be connected to tissue by soft tissue anchors. Examples of soft tissue anchors used by applicant include those used in the MiniArc® mesh sling. It is also possible to suture the arm portions 26a and 26b in place.

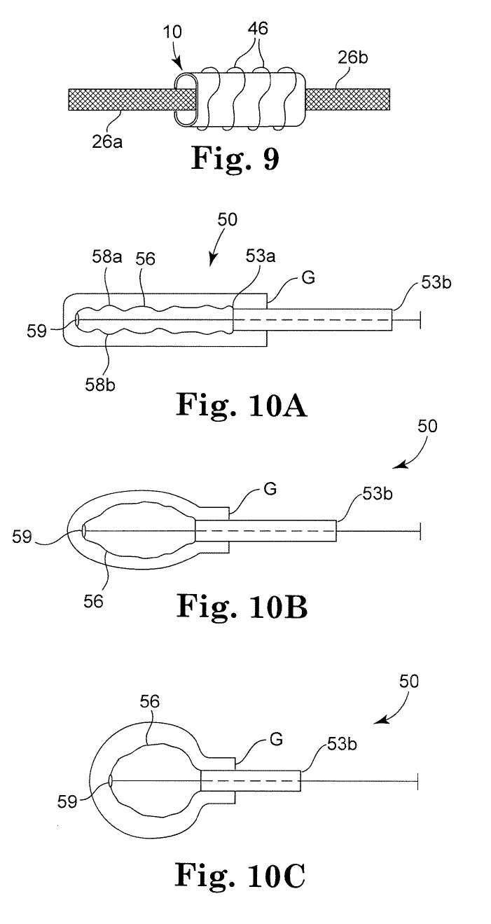

Turning now to FIG. 9, the support member 10 can be coiled or folded and bound by one or more sutures 46 tied around at least a central portion of the insertion tool 12. The arm portions 26a and 26b can have couplers that can be coupled to needles or other delivery devices. The delivery devices can be used to insert the arm portions 26a and 26b into one or more incisions. Once the central portion of the insertion tool 10 is in a therapeutic location a physician can cut the sutures 46, thereby, allowing the resilient members 40 to move the support member 10 from its folded or coiled state to its natural state, which can be flat or slightly coiled. The sutures 46 can also be tied with an end of the sutures 46 extending out of the incision. Pulling on the suture 46 extending out of the incision can cause the sutures 46 tied around the support member 10 to be untied, thereby, allowing the support member 10 to uncoil.

If tissue needs to be dissected prior to implantation of the support member 10 a spreader 50, as illustrated in FIGS. 10A-10C, can be used. The spreader 50 comprises a handle 52 having a first end 53a and a second end 53b. The handle 52 can have size and shape that permits it to be easily inserted into an incision. In an example embodiment, the handle 52 is generally elongate but other shapes and configurations are also contemplated. The handle 52 can comprise a rigid, semi-rigid or pliable material. The pliability of the handle 52 can permit it to be bent in order to more easily access a particular therapeutic location. The handle 52 can also have different cross sectional shapes. For instance, the handle 52 can have a generally round cross section, a generally flat cross section, a concave or convex cross section, or any variations thereof.

A dilator 56 used to dilate tissue in the pelvic cavity can be operatively coupled to the first end 53a of the handle 52. The dilator 56 can comprise one or more tissue dissecting portions 58a and 58b. Each of the tissue dissecting portions 58a and 58b is coupled to the first end 53a of the handle 53a at one end and to a connector 59 at another end such that the dilator 56 generally forms a loop. In one example embodiment, the tissue dissecting portions 58a and 58b can comprise a material such as a polymer, nitinol or a low plastically deforming metal. Other materials can also be used and should be considered to be within the spirit and scope of the invention.

A dilator actuator 60 can be slidably disposed in or on the handle 52 and operatively coupled to the connector 59. The dilator actuator 60 can comprise an elongate rod 62 that is capable of traveling axially through the handle 52. The dilator actuator 60 can comprise a material similar to the handle 52. In one embodiment, the dilator actuator 60 can comprise a polymer, nitinol or low plastically deforming metal that permits it to be deformable in the shape of the handle 52.

The dilator 56 has a closed position to permit insertion of the dilator 56 into and out of an incision. The closed position, as illustrated in FIG. 10A, has the tissue dissecting portions 58a and 58b generally parallel to each other and lying generally along an axis of the handle 52. The dilator 56 also has an open position, whereby, movement from the closed position to the open position dissects tissue. The open position, as illustrated in FIG. 10C, has the tissue dissecting portions 58a and 58b positioned generally apart from each other. In one example embodiment, the tissue dissecting portions 58a and 58b can form an opened loop, a square loop, a triangle loop, or any shape in the open position.

The dilator actuator 60 can be used to move the dilator 56 between the open and closed positions. When the dilator actuator 60 is inserted into the handle 52 it pushes against the connector 59 moving it axially away from the handle 52 and drawing the tissue dissecting portions 58a and 58b toward each other as illustrated in FIG. 10A. When the dilator actuator 60 is withdrawn from the handle 52 it pulls the connector 59 toward the first end 53a of the handle 52 which causes the tissue dissecting portions 58a and 58b to move away from each other as illustrated in FIG. 10C.

As particularly illustrated in FIGS. 10A-10C it can be seen that the dilator 56 can be inserted into an incision G prior to moving the dilator 56 from the closed position toward the closed position. As the dilator actuator 60 is withdrawn from the handle 52 it moves the dissecting portions 83a and 83b away from each other. As the dissecting portions 83a and 83b move away from each other they dissection the tissue and create an implantation site for the support member 10.

In one embodiment of the invention, the dissecting portions 83a and 83b can comprise wires or blades that are able to cut various forms of tissue. The dissecting portions 83a and 83b can also travel in a particular predetermined plan as they move between the open and closed positions. This permits a physician to carefully control the area of dissection. One or more markers can be displayed on the handle 52 to identify the plane of dissection to a physician. If a larger area needs to be dissected a physician can rotate the handle 52 to alter the plane of dissection.

Another example embodiment of the invention includes having a dilator 56 that has multiple dissection portions radiating in a generally spherical shape. The spherical shape permits a physician to create a generally spherical cavity. It is also possible to have a dilator 56 that is generally concave to permit a physician to create a concave or convex cavity. A motor can also be operatively coupled to the dilator 56 to mechanized dissection.

In use, an incision can be made in a patient and a therapeutic location identified. The support member 10 can be wrapped or coiled about the tissue support portions 18 of the insertion tool 12. The tissue support portions 18 can be placed in the closed position prior to inserting the insertion tool 12 into the incision. Once the tissue support portions 18 are disposed in the therapeutic location, the physician can move the handle portions 16 to move the tissue support portions 18 from the closed position toward the open position. As the tissue support portions 18 move toward the open position the support member 10 uncoils and is laid flat or in the predetermined configuration of the support portions 18.

The support member 10 can also be pliable and capable of being wound, coiled, or curled about the insertion tool 12 prior to insertion into an incision. In this particular example embodiment incisions are made and the insertion tool 12 with the support member 10 coiled or wrapped around it are inserted into the incisions. Actuators 32a and 32b of insertion tool 12 preferably extend out of incisions E and F. A physician then moves the actuators 32a and 32b causing the expansion portions 33a and 33b to move from the closed position toward the open position. As the expansion portions 33a and 33b move toward the open position they can dilate the tissue, if not done earlier, uncoil the support member 10 and permit it to lay flat or against the desired organ or tissue. Once the physician has determined that the support member 10 is properly positioned the actuators 32a and 32b are moved away each other, thereby, causing the expansion portions 33a and 33b to move toward the closed position. The insertion tool 12 is then removed and the incisions closed.

In particularly tight therapeutic locations, the support member 10 can be coiled or folded and placed into a sheath 42. The sheath 42 can then be inserted into an incision. If the therapeutic location is difficult to reach one or more couplers 44 can be disposed on or operatively coupled to the sheath 42 for coupling to a needle or similar deliver device to facilitate passage through the incision and into a therapeutic location for implantation. Once in place the sheath 42 can be removed exposing the support member 10. The resilient members 40 then cause the support member 10 to uncoil or unfold toward its natural state, which could be flat or slightly coiled. If the support member 10 has arm portions 26a and 26b they can be anchored to tissue by soft tissue anchors.

If the physician needs to conduct some tissue dissection prior to implantation of the support member 10 they can use the dilator 56. The dilator 56 can be placed in the closed position and the tissue dissection portions 58a and 58b inserted into the incision. The physician can then move the dilator actuator 60 out of the handle 52, thereby, causing the tissue dissecting portions 58a and 58b to expand or move to the open position. The physician can continue moving the dilator actuator 60 in and out to dissection the tissue.

Fecal incontinence can be treated by using any of the above embodiments. The support member 10 or mesh supported with mandrels or other rigid or resilient members 40 that can cause the support member 10 to hook onto and wrap either side of the levator. By applying a torsional force to the levator it can be distorted such that the effective distance between two sides of the levator are shortened, and raised, thereby providing support to the pelvic organs.

Fecal incontinence can also be treated by introducing an energy source, such as RF or laser, or chemical via a hypo-needle inserted through the transobturator foramen for the purpose of causing a catalyst which would promote tissue regrowth into a bioresorbable mesh or bolster. Once the support member 10 is implanted, the chemical promotes tissue regrowth into the support member 10 if manufactured from a mesh material or bolster. Over time a bioresorbable the support member 10, if used, can be absorbed by the body leaving new tissue to support the levator. It is also possible to use cell-based nano technology to promote tissue ingrowth into the support member 10 (mesh or bolster). Any substance that can cause hemostasis to remodel the levator can be used either individually or in combination with a mesh or bolster.

Another effective treatment for fecal incontinence is the use of electrodes or other energy transmitting devices mounted onto or fixed to a mesh or sling that is implanted into a patient to stimulate the pelvic floor, levator muscles for remodeling and/or visceral organs, rectum, bowel and/or pelvic floor to obtain continence and reduce or eliminate perineal descent or levator ballooning.

In order to treat fecal incontinence in another example embodiment, the support member 10, which can comprise a mesh sling, is implanted by making one or more incisions in the posterior region of the patient lateral the coccyx. The support member 10 can wrap around the coccyx, sacrum and other anchor points in the base of the spine, the support member 10 could also fixate into the spine via bone anchor technology. Such a fixated support member 10 could traverse around and support the rectum and anal sphincters similar to obturator approaches. In order to treat fecal incontinence a support member 10 can be implanted by making one or more incisions in the posterior region of the patient lateral the coccyx. The support member 10 can wrap around the bony structure to provide strong support for tensioning the levator.

Another embodiment for effecting paravaginal repair, is to make as least one incision or puncture at the obturator (or alternatively, at each obturator) and pass a needle or introducer with a support member 10 attached thereto (with or without a plastic sheath) and anchor the end of the sling (with or without a physical tissue anchor) to the tissue or muscle near or at the levator ani muscle(s) and then pull the sling up through the obturator to effectively pull up the tissue and anatomy back to its original position or orientation. In a related embodiment, the sling may be a short mesh pieces, with or without anchors, that is also embedded in pelvic tissue or muscle and its pulled part of the way up to the obturator, anchored at the obturator but not through the obturator, thereby avoiding a single or more incisions in the vaginal wall or perineal floor of the patient. The repair is done completely from above without disturbing the lower portion of the patient's anatomy or pelvic area.

It is also possible to repair a paravaginal defect by using the white line as a landmark for securing the mesh as well.

It is also possible to repair a paravaginal defect by attaching a first magnetic member on pubic bone and a second magnetic member on a portion of the levator muscle. The first and second magnetic members are magnetically attractable when brought in generally close proximity to each other. The paravaginal is corrected by allowing the first and second magnetic members to be magnetically connected. In another embodiment, the first magnetic member can have a plurality of spaced receiving portions for receiving the second magnetic member. A physician is able to adjust the tension of the muscle by placing the second magnetic member in different receiving portions. It is also possible that a physician could use a third magnetic member that is magnetically attracted to the second magnetic member to move the second magnetic member from outside of the patient.

Fecal incontinence can also be treated by injecting a chemical that causing tissue scarring and shrinkage into various parts of the levator muscle(s) that causes it to contract and remain contracted, thereby pulling up the various pelvic tissue and organs

In another embodiment, the chemical can be injected into the levator muscle to cause contraction while a support member 10 or coiled mesh or mesh-spring is implanted into or onto the levator muscle. By pre-contracting the muscle the mesh or mesh-spring, becomes fixated to the contracted muscle and reduces the amount of levator muscle is able to relax, thereby lifting the pelvic floor.

Coiled mesh, mesh slings, or springs having retracting or coiling abilities can be implanted in or connected to the levator muscle to cause lifting of the levator muscle. In one embodiment, the coiled mesh can have first and second anchors disposed on its opposite ends. The first anchor can be fixated to tissue such as the levator tissue. The coiled mesh can then be pulled or lengthened and the second anchor fixated into tissue such as the levator muscle. The coiled mesh or spring is then permitted to coil such that the first and second anchors are drawn closer together. The coiling action of the mesh or spring causes the levator muscle to lift, thereby correcting any paravaginal defect. A gun type delivery device could be used to deliver one or more of the coiled meshes or springs into the patient. In one embodiment, the first anchor can protrude through an end of the gun. The gun is then inserted into the tissue. The user then depresses the trigger to a first position, wherein the first anchor is ejected from the gun and into the tissue. The user can then begin to retract the gun, thereby causing coiled mesh or spring to extend out of the gun. As the user retracts the gun the second anchor abuts an end of the gun and the coiled mesh or spring is uncoiled. The user is then able to depress the trigger to a second position wherein the second anchor is ejected from the gun and into the tissue. The natural tendency of the coiled mesh or spring is to coil or retract. As the coiled mesh or spring retracts it lifts the levator tissue.

A V-shaped frame can be constructed by affixing nitinol or other relatively rigid material between each ischial spine or sacrospinous ligament, and advancing each to the sacrum (using our Straight-In device, laparoscope, or robot), caudal to the levators. With this relatively rigid structure in place above the levator, a nitinol ring or coil that applies outwards pressure (i.e., expands in circumference once placed), encompassing a larger area than the “V” can be advanced under the levators, but pressed up and caudal to the V-frame, but from below the levator (thereby forcing the levators to distort above and around the V-frame). The outward pressure generated by the ring or coil will apply force to the levators, pushing them around the V-frame thereby causing lift.

Levator ani muscle restoration can be achieved by using at least one mesh sling having at least one anchor fixed to an end with the other end attached to or near the pubic bone. In another embodiment, the surgical implantation of the fecal mesh sling uses a c-needle with a reusable or pushable dilator to allow the sling to be moved from one perineal incision to the other, each incision being located on either side of the rectum or anus. Example of dilation tools and techniques between two (or a single perineal incision used to tunnel up to the obturators) are illustrated and discussed in Appendix A, which is herein incorporated by reference in its entirety.

Material Changes include better resistant; embedded mesh with tines or studs; an umbrella or anchor shaped piece that resists movement through the patient's adipose tissue, preventing it from moving inferiorly in the patient. Extra dilators on the support member 10 or mesh can be used to allow for easier passing through the patient.

The above aspects of the invention provide the desired surgical devices and procedures to treat fecal incontinence and prolapse without having to do extensive tissue dissection. The various embodiments and surgical treatments disclosed above are applicable to both female and males and can be used to cure perineal and paravaginal defects. In addition, various forms of tissue ablation (cryo, thermal, RF (microwave, radio frequency) and laser therapies) can be use to promote scarring and shrinkage in tissue either alone or in combination with the invention, slurries or gels or other chemical compositions or implants.

The following patents, application and publications are herein incorporated by reference in their entirety US 2005/0049648, US 2002/0161382, US 2002/0007222, U.S. Pat. No. 6,612,977, U.S. Pat. No. 6,911,003, US 2007/0021650, WO 2006/0690078, U.S. Pat. No. 6,506,190, US2004/0039453, US2005/0250977, US2007/0260288, U.S. Pat. Nos. 6,554,824; 6,554,825; 6,986,764; 5,151,909; 5,243,615; 6,096,052; 6,432,116 and 7,073,504 and U.S. Patent Publications 2004/0087995; 2005/0070938 and 2005/0131431. In addition: WO/02-091935/PCTUS01/51646.

We claim:

1. A pelvic implant assembly comprising:
a support member comprising a panel; and
an insertion tool comprising an implant support portion for supporting the support member, the support member being coupled to and extending between the implant support portions prior to insertion into an incision, wherein the implant support portions move away from each other and spread out the support member.

2. The pelvic implant assembly of claim 1, wherein ends of the implant support portions are generally pointed to ease insertion into an incision.

3. The pelvic implant assembly of claim 1, further comprising retaining members operatively coupled to each implant support portion, wherein ends of the support member are coupled to the retaining members prior to insertion.

4. The pelvic implant assembly of claim 3, wherein the retaining member comprise at least one selected from the group comprising a recess or channel extending into each of the implant support portions, clips, or rollers.

5. The pelvic implant assembly of claim 1, wherein each of the implant support portions include channels or recesses extending therein that permit the support member to be generally flush with an outer surface of the implant support members.

6. The pelvic implant assembly of claim 1, wherein the support member comprises a central portion with one or more appendages and extending away from the central portion, wherein the central portion can be wrapped or coiled about the implant support portions.

7. The pelvic implant assembly of claim 6, wherein at least one anchor is disposed to at least one end of the support members or appendages for anchoring into tissue.

8. The pelvic implant assembly of claim 1, wherein the insertion tool further comprises a handle to control the implant support portions.

9. The pelvic implant assembly of claim 1, wherein the insertion tool comprises a plurality of interconnected segments or linkages, the segments comprising at least one actuator portion operatively coupled to at least one expansion portion, wherein the support member is coupled to the expansion portion prior to insertion into an incision and wherein the expansion portion expands creating a cavity and properly positioning the support member.

10. The pelvic implant assembly of claim 9, wherein the insertion tool further comprises joints operatively connecting the segments to the at least one expansion portion to move relative to the at least one actuator portion.

11. The pelvic implant assembly of claim 1, further comprising resilient member disposed on the support member to move the support member to a predetermined shape.

12. A pelvic implant assembly, comprising:
an insertion tool comprising a plurality of segments or linkages, the segments comprising at least expansion portions operatively disposed between at least two actuator portions, the expansion portions being coupled to the actuator portions by joints; and
a support member comprising a panel having a pocket formed thereon to receive at least a portion of the insertion tool, the support member being folded over the expansion portion of the insertion tool, wherein expansion of the expansion portions spreads or uncoils the support member after it is implanted

13. The pelvic implant assembly of claim 12, wherein the expansion portions have a plurality of intermediate joints capable having an expansion portion a shape selected from the group comprising a regular hexagon, irregular hexagon, circle, or oval.

14. The pelvic implant assembly of claim 12, wherein the pocket is formed by tying two spaced apart lengths of sutures generally parallel to each other on the panel.

15. The pelvic implant assembly of claim 12, wherein the pocket is formed by heat setting or coupling a piece of material to the panel.

16. The pelvic implant assembly of claim 12, further comprising at least one resilient member disposed on the support member to cause the support member to return to a predetermined shape.

17. The pelvic implant assembly of claim 16, further comprising a sheath that can encase the support member as it is being inserted into an incision, wherein after it is removed the support member returns to a predetermined shape.

18. The pelvic implant assembly of claim 12, further comprising at least one suture that bounds the support member to the insertion tool, wherein removal of the suture permits the support member to return to a predetermined shape.

19. The pelvic implant assembly of claim 12, further comprising a spreader to dissection tissue, the spreader comprises:
a handle having a first end and a second end;
a dilator operatively coupled to the first end of the handle, the dilator having a tissue dissecting portion that moves between an open and closed position to dissect tissue; and
a dilator actuator operatively coupled to the dilator to move the dilator between the open and closed positions.

20. The pelvic implant assembly of claim 19, further comprising markings on the handle to identify a plane of dissection to a physician.
